(19) United States
(12) Patent Application Publication
(10) Pub. No.: US 2024/0087735 A1
(43) Pub. Date: Mar. 14, 2024

(54) MOVEMENT SENSOR FOR PATIENTS UNDERGOING MEDICAL PROCEDURES

(71) Applicant: Alexander Markovic, Fort Collins, CO (US)

(72) Inventor: Alexander Markovic, Fort Collins, CO (US)

(21) Appl. No.: 18/228,992

(22) Filed: Aug. 1, 2023

Related U.S. Application Data
(63) Continuation-in-part of application No. 16/997,327, filed on Aug. 19, 2020, now abandoned.
(60) Provisional application No. 62/936,547, filed on Nov. 17, 2019.

(51) Int. Cl. G16H 40/63; A61B 6/00; A61B 6/04

(52) U.S. Cl. CPC G16H 40/63; A61B 6/04; A61B 6/463; A61B 6/465; A61B 6/467; A61B 6/56

(57) ABSTRACT

An apparatus for monitoring patient movement during a medical procedure comprises a single calibrated wireless sensor module responsive to movement using an inertial measurement unit measuring angular pitch changes with an internal microprocessor calculating angular pitch data over time. A patient movement monitor displays visual data graphically depicting movement within one or more movement parameters required to execute a medical procedure and visible to a patient during the medical procedure. A sensor control and monitoring unit controls the wireless sensor module and to receive movement data as calculated angular pitch data over time, processes the movement data by averaging pitch data over a number of data points and generate a graphic output on an associated display, and displays the processed movement data as a graphic indicating movement within at least one movement parameter on both a patient movement monitor and a work station display, providing movement feedback to the patient.

### CROSS REFERENCE TO RELATED APPLICATIONS

This application claims benefit of U.S. Provisional Application 62/936,547 filed Nov. 17, 2019, which is incorporated herein. This application is also a Continuation-in-Part (CIP) of U.S. application Ser. No. 16/997,327 filed Aug. 18, 2020 and claims benefit thereof.

### FIELD OF INVENTION

The present invention relates to the field of wireless medical positional monitoring system, more particularly to a wireless positional monitoring system providing position and movement feedback to patients and equipment operators for radiological treatments and imaging such as for radiotherapy and imaging procedures.

### BACKGROUND

Maintaining patient positioning and minimizing body movement to within acceptable parameter presents an ever-present and prevalent problem in thoracic or other radiological imaging, Magnetic Resonance Imaging (MRI), and oncological radiotherapy. Most patients undergoing radiation treatment or diagnostic imaging cannot maintain the necessary body position and minimize movement to ensure optimal exposure of tumors to radiation emissions without at least some degree of training. Moreover, many patients exhibit excessive movements outside acceptable treatment parameters, adversely affecting the level of received radiation, during at least some portion of their treatment regimen. This can be especially problematic in lung or breast cancer patients, who can be asked to hold their breaths for long periods of time that patients can find particularly difficult or impossible to accomplish.

Prior art efforts at position monitoring using wireless sensors have been directed at improving operator observation of patients during imaging or treatment. None of the prior art implementations have been utilized to provide a monitoring system usable by both an equipment operator and a patient. Currently, other products on the market are based on camera/optical technology which are usually prohibitively expensive. Furthermore, these systems need to be mounted to the ceiling of the radiation treatment room and thus are not portable. The proposed technology uses inexpensive electronic components making it more accessible to customers and clinics that cannot afford the camera-based systems. Due to its portability, the proposed system has the additional advantage of being able to be used as a coaching tool in exam rooms. Another advantage over camera-based systems is that this system does not require the patient to be uncovered during treatment, which addresses a modesty issue for many patients.

A system that can easily monitor the movement of a patient and display the data in an easily understood format to inform a patient that their movements remain within acceptable parameters or exceed parameters could greatly aid in keeping patients in position. A double display output for both the patient and the medical equipment operator to monitor movement can offer considerable flexibility of use and improve patients compliance. A small and simple movement sensor can further improve ease of use and patient comfort.

Based on the foregoing, there is a need in the medical imaging and radiology oncology treatment arts for a method and process facilitating increased compliance by patients to keep body movement within acceptable parameters. A simple, easy to use sensor and data monitor and display system can enable individual patients to comply with instructions to minimize movement and remain positioned correctly for medical procedures.

### SUMMARY

In an embodiment, a smartphone, tablet, or other mobile, semi-mobile, or fixed patient feedback device attached to a radiological device or MRI can be programmed and configured to provide visual feedback to a patient or machine operator while undergoing imaging or treatment. This real time monitoring is an inherent requirement to successful oncology treatment to ensure delivery of the specific therapeutic radiation dose to a desired treatment site during the treatment. A wireless, programmable “smart” inertial measurement unit (IMU), which can include an accelerometer, a gyro, or a magnetometer sensor, can operate software to translate movement of the sensor into a data stream reflecting angular pitch movement of the patient. The “smart” IMU can include an accelerometer, a gyro, or a magnetometer sensor that transmits the data stream by radio, Bluetooth, or other wireless transmission protocol to an operator's work station as well as the configured patient feedback device to receive and process the data.

In a specific application, in radiation oncology, patients must hold still during their treatments in order for the radiation beam to hit the correct location. Their breathing motion must be monitored to ensure they are in the correct breathing phase prior to delivery of the treatment. This system monitors the position of a patient during treatment (i.e., in real time) to ensure they are being irradiated correctly. It is also considerably less expensive than current technology, because it uses simple components to implement a dual graphic output of movement data to both the technician administering treatment and the patient on separate displays.

This first part of this system can consist of a small 3×3×5 cm electronic module box with a power switch. Inside the box is a circuit containing four major components: a microcontroller unit, a radio (e.g., RFM69 radio), an IMU (e.g., an accelerometer, a gyro, or a magnetometer) and a rechargeable battery (e.g., 150 mAh rechargeable LiPo battery). This is the sensor unit that is attached to the patient's body. The second part consists of a 12×12×4 cm case with a 7×5 cm touchscreen. The electronics inside the case can consist of a circuit board, a radio (e.g., RFM69 radio), a microcontroller, and a rechargeable battery (e.g., 2500 mAh rechargeable LiPo battery) and can be used to provide visual feedback to the patient in real time during treatment.

This system is based on a small electronic module, or wireless sensor, that can be placed anywhere on the patient's body. It then monitors the movement/position of the patient with an IMU and wirelessly relays the data to a receiver module that displays the processed data on a screen. The data can be sent by the module over 933 MHz RF at a rate of 14 HZ to the portable receiver module which displays the information on a touchscreen for a patient or the technologist to view. The screen can display the position information as either a graph or a moving bar and can easily be mounted over the patient's head and used to provide them with helpful feedback regarding their position during treatment. The screen and IMU modules are portable and thus can be used in locations other than the treatment room for teaching and coaching purposes.

A system for monitoring patient movement during a medical procedure requiring minimal movements, comprising a wireless sensor module responsive to movement incorporating a microprocessor and executable computer code stored in memory and an IMU connected to the microprocessor sensitive to angular pitch changes and attached to a patient. A patient movement monitor incorporating a microprocessor and executable computer code stored in memory and a display displaying processed patient movement data including visual data graphically depicting movement within one or more movement parameters required to execute the medical procedure and visible to the patient during the medical procedure interfaced with the wireless sensor module. A sensor control and monitoring unit incorporating a microprocessor and executable computer code stored in memory and a display in wireless communication with the wireless sensor module. The sensor control and monitoring unit can remotely interface with the wireless sensor module to control the wireless sensor module and to receive movement data, process the movement data, and display the movement data as a graphic indicating movement within at least one movement parameter.

An apparatus for monitoring patient movement during a medical procedure, comprising a wireless sensor module responsive to movement using an IMU measuring angular pitch changes. A patient movement monitor that comprises a display with processed patient movement data including visual data graphically depicting movement within one or more movement parameters required to execute the medical procedure and visible to the patient during the medical procedure interfaced with the wireless sensor module. A sensor control and monitoring unit remotely interfaced with the wireless sensor module to control the wireless sensor module and to receive movement data, process the movement data, and display the movement data as a graphic indicating movement within at least one movement parameter. The patient movement monitor provides a movement graphic indicating compliance with a maximum allowable movement parameter.

An apparatus for monitoring patient movement during a medical procedure, comprising a single calibrated wireless sensor module comprising an internal microprocessor with a CPU coupled to an associated first memory and first radio responsive to movement using an inertial measurement unit measuring angular pitch changes consisting of measuring gravitational data generated by the inertial measurement unit with the internal microprocessor calculating angular pitch data over time; a patient movement monitor comprising a display with processed patient movement data displayed including visual data graphically depicting movement within one or more movement parameters required to execute the medical procedure and visible to a patient during the medical procedure, said patient movement monitor interfaced with the wireless sensor module; a sensor control and monitoring unit comprising a microprocessor with CPU, an associated second memory, second radio, and associated work station display remotely interfaced with the wireless sensor module to control the wireless sensor module and to receive movement data as calculated angular pitch data over time, process the movement data by averaging pitch data over a number of data points and generate a graphic output on an associated display, and display the processed movement data as a graphic indicating movement within at least one movement parameter, said sensor control and monitoring unit connected to the patient movement monitor and the work station display; wherein the sensor control and monitoring unit processes the movement data to generate a real time graphic data output providing a graphical display feedback to the patient movement monitor to the patient showing processed movement data in real time of relative patient movement depicting a movement threshold that meets acceptable movement parameters for the medical procedure along with a secondary indicator that patient movement is not excessive and also meets those acceptable movement parameters and/or 1) a breath hold duration of time that the patient has been able to hold their breath and/or 2) time the patient has maintained their body position within those the acceptable movement parameters, whereby said patient movement monitor and provides a visual feedback to the patient that patient movement meets or exceeds allowable acceptable movement parameters for the medical procedure, both said patient movement monitor and work station display displaying the real time graphic data output.

### Advantages

1. Improved ability for patients to monitor their movements during medical procedures using visual feedback.
2. Improved monitoring by a technician of patient movement and position during medical procedures.
3. Improved ease of monitoring.
4. Allows feedback to patients and permits training of patients for improved compliance with movement restrictions and maintaining position within position constraints and parameters.
5. Cheaper and easier to implement.
6. Does not require expensive hardware or computer upgrades or modifications.
7. Portable.
8. Improved modesty and comfort for patients (no requirement for treatment of uncovered patients).

The foregoing, and other features and advantages of the invention, will be apparent from the following, more particular description of the preferred embodiments of the invention, the accompanying drawings, and the claims.

### BRIEF DESCRIPTION OF THE DRAWINGS

For a more complete understanding of the present invention, the objects and advantages thereof, reference is now made to the ensuing descriptions taken in connection with the accompanying drawings briefly described as follows.

FIG. 1 is a schematic block diagram of components of the mobile computer/communication devices configurable with patient movement module computer code, according to an embodiment of the present invention;

FIG. 2A is a schematic diagram of the major components of a patient movement monitoring system during a medical procedure, according to an embodiment of the present invention;

FIG. 2B is a front schematic diagram of FIG. 2A of the major components of a patient movement monitoring system during a medical procedure, according to an embodiment of the present invention

FIG. 3 is a schematic view of a medical equipment work station, according to an embodiment of the present invention;

FIG. 4 is a schematic view of electronics of a wireless sensor module, according to an embodiment of the present invention;

FIG. 5 is an exemplary exterior view of a wireless sensor module, according to an embodiment of the present invention;

FIG. 6 is an exemplary view of a display showing processed movement data collected from the wireless sensor module, according to an embodiment of the present invention;

FIG. 7 is an exemplary view of a display showing a second view processed movement data collected from the wireless sensor module, according to an embodiment of the present invention;

FIG. 8 is an exemplary view of a display on a sensor control unit showing a display with control inputs, according to an embodiment of the present invention;

FIG. 9 is a second exemplary view of a display on a sensor control unit showing a display with control inputs, according to an embodiment of the present invention;

FIG. 10 is an exemplary process flowchart for breathing monitoring in a wireless sensor module, according to an embodiment of the present invention; and

FIG. 11 is an exemplary process flowchart for the sensor monitor control unit interfaced with a wireless sensor module, according to an embodiment of the present invention; and

FIG. 12 is an exemplary process flowchart showing the general overall process for using the sensor control and monitoring unit interfaced with the wireless sensor module to generate and output a patient feedback graphic onto a patient monitor, according to an embodiment of the present invention.

### DETAILED DESCRIPTION OF PREFERRED EMBODIMENTS

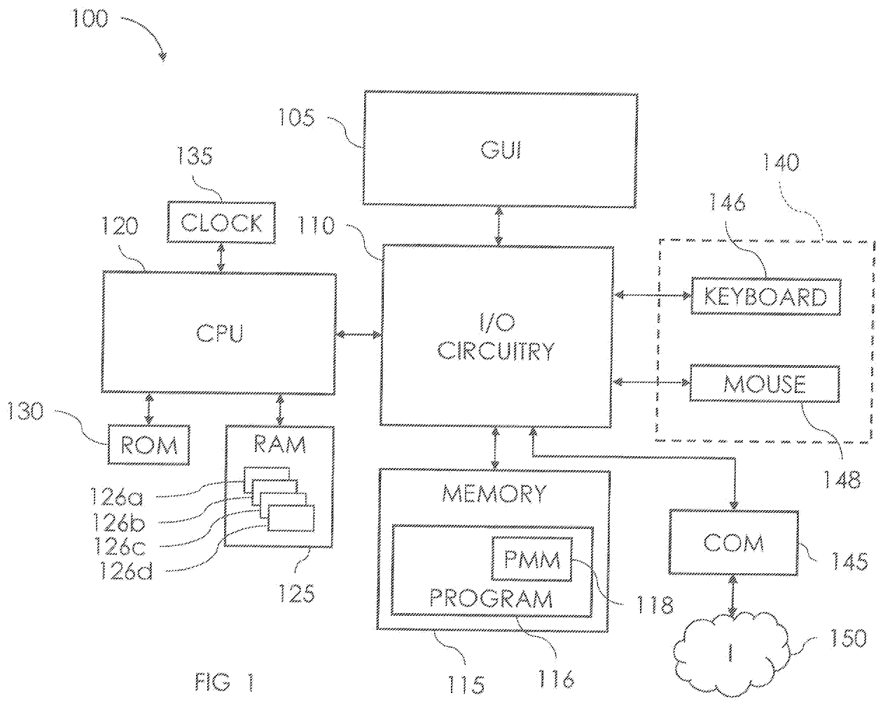

Preferred embodiments of the present invention and their advantages may be understood by referring to FIGS. 1-12, wherein like reference numerals refer to like elements.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present application or of any further application derived therefrom.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

Headings provided herein are for convenience and are not to be taken as limiting the disclosure in any way.

The enumerated listing of items does not imply that any or all of the items are mutually exclusive, unless expressly specified otherwise.

The terms “a”, “an” and “the” mean “one or more”, unless expressly specified otherwise.

Devices or system modules that are in at least general communication with each other need not be in continuous communication with each other, unless expressly specified otherwise. In addition, devices or system modules that are in at least general communication with each other may communicate directly or indirectly through one or more intermediaries.

The computer memories in the various disclosed devices may store computer executable instructions. Each disclosed computer/communication device such as computer, a server, a system node, a smart phone, a tablet, or similar device able to execute computer code and/or process digital, electronic data may execute computer executable instructions. The computer executable instructions may be included in computer code. The computer code may be stored in the various device memories. The computer code may be written in any computer language comprising the prior art. The memory may be a non-transitory tangible storage media such as a compact disk (CD), flash drive, floppy disk, micro disc card, hard drive, solid-state drive (SDD), or similar type of storage device or media capable of storing computer code (e.g., software code) in a non-transitory and computer-accessible state. Sophisticated computer apps have increasingly become available, with downloaded executable software code (e.g., the Apple® Store) providing for configuring a mobile device, such as a smart phone or tablet, to perform a plethora of functions.

The computer code may be logic encoded in one or more tangible media or one or more non-transitory tangible media for execution by the processor in the devices. Logic encoded in one or more tangible media for execution may be defined as instructions that are executable by the processor and that are provided on the computer-readable storage media, memories, or a combination thereof. Logic may include a software controlled microprocessor, an application specific integrated circuit (ASIC), an analog circuit, a digital circuit, a programmed logic device, a memory device containing instructions, and the like. The instructions may be stored on any computer readable medium comprising the prior art from which a computer, a processor, or other electronic device can read. This may include a computer data disk or the like storing computer code that can be used to configure a memory associated with a computer, a processor, or other electronic device.

The processor may include a general processor, digital signal processor, ASIC, field programmable gate array, analog circuit, digital circuit, central processing unit (CPU), micro-processor unit (MPU), micro-controller unit (MCU), combinations thereof, or other now known processor. The processor may be a single device or combinations of devices, such as associated with a network or distributed processing. The processor may be responsive to or operable to execute instructions stored as part of software, hardware, integrated circuits, firmware, micro-code or the like. The functions, acts, methods or tasks illustrated in the figures or described herein may be performed by the processor executing instructions stored in the memory.

A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

The present invention will now be described in detail with reference to embodiments thereof as illustrated in the accompanying drawings.

As depicted in FIG. 1, a schematic block diagram 100 shows the internal components of a computer smartphone, laptop, desktop, or tablet (or similar computer/communication device), any of which can be programmed and configured with appropriate software to implement the patient's visual feedback monitor (PM 240) and/or the system interface and control unit (SCMU 330), that can comprise a display 105 comprising a touch sensitive screen functioning as a graphical user interface (GUI), input/output (I/O) circuitry 110 for processing and routing input and output data, memory 115 storing digital data, which can include computer programs (in the form of executable program code) 116, including the patient movement monitor application (PMM) 118 of the invention, a central processing unit (CPU) 120, with associated Random Access Memory (RAM) 125 consisting of RAM modules 126a, 126b, 127c, and 126d, Read Only Memory (ROM) 130, a clock 135 regulating the function of the device, and external hardwire 140 that may be attached, such as a keyboard 146 or a mouse 148, which also can serve as a GUI. However, typically touch inputs on the touch sensitive display/GUI 105, which may also include virtual graphical analogues of the mouse 148 and keyboard 146. Finally, the device can include a communication module (CM) 145 for accessing the Internet 150 and transmitting or receiving data between the device and external data sources, such as a computer server hosting an Internet accessible website. CM 145 can support communication over a cellular mobile network, WAN, WiFi, Bluetooth, or similar wireless connection to interface or communicate other devices. Using these communication resources, CM 145 can access and communicate over the Internet 150 or use a short range wireless connection to a nearby device.

In operation, the CPU 120 can access through I/O circuitry 110 memory 115 storing programs 116 including the SB 118 of the invention. The CPU 120 can execute the programs 116 as well as processing any stored digital data in memory 115. The CPU 120 can also operate to process data received or transmitted via communication module 145 using I/O circuitry 110. The CPU 120 can process received data from or transmitted to communication module 145 as well as data manipulation inputs from the GUI screen 105 of the tablet or exterior hardware 140. The touch sensitive screen forming GUI 105 can receive data inputs reflecting scrolling or swiping movements of a user's finger, which the CPU 120, utilizing executed programs from memory 115, uses to process data received from communication module 145 to display that data reflecting those scrolling and swiping inputs from a user on screen 105.

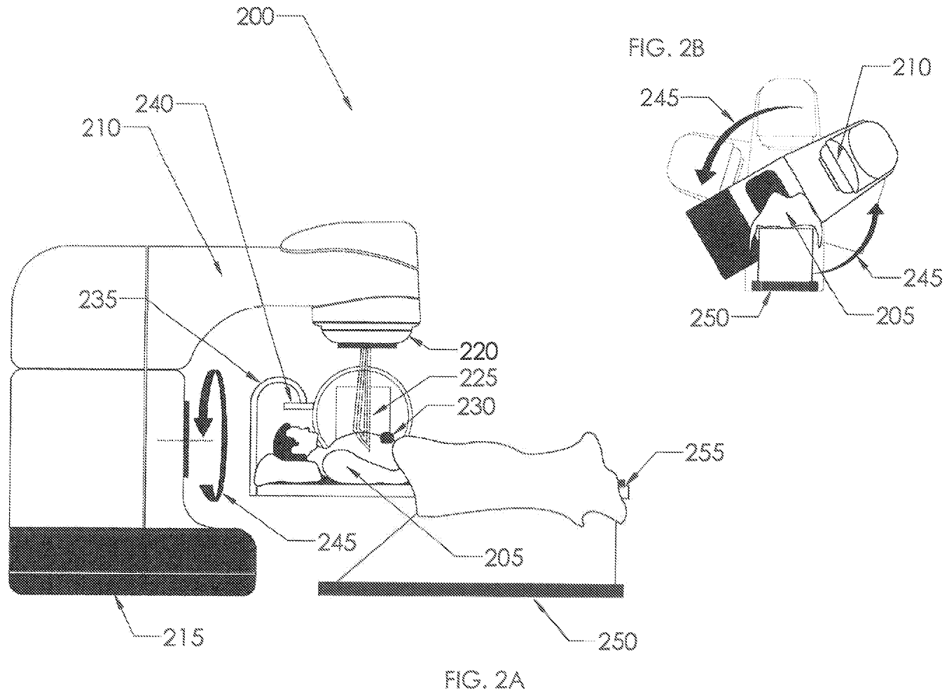

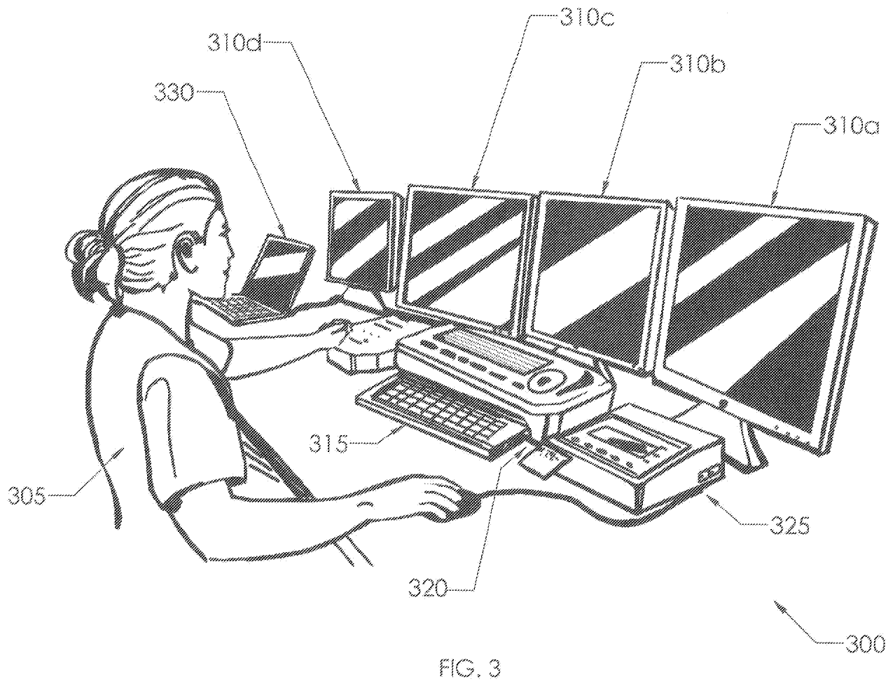

FIGS. 2A, 2B, and 3 depict the elements of a radiological oncology treatment system used in an exemplary embodiment of the invention. FIGS. 2A and 2B depict a radiological oncology treatment system setup 200 for a patient 205. Base 215 attaches to oncology treatment unit 210. In an embodiment, oncology treatment unit 210 can rotate around rotational vector 245.

The oncology treatment unit 210 generates a therapeutic radiation beam 225 from a radiation beam generator assembly 220. The patient 205 must maintain position under the radiation beam generator assembly 220 within acceptable movement parameters for a specific time in order to effectively treat a tumor with the required therapeutic dose of radiation delivered by radiation beam 225. Wireless sensor module (WSM) 230 attached to the patient 205 includes, in an embodiment, a circuit containing four major components: a microcontroller unit, a RFM69 radio, an IMU, and a 150 mAh rechargeable LiPo battery. Although WSM 230 as depicted has been attached to the patient's chest, it may also be attached to the abdomen, head, shoulders, or other region of the patient's body. The body may be either covered or uncovered. In an embodiment, the WSM 230 can be attached using a medical adhesive. Furthermore, in an embodiment, the sensor can detect movement in multiple areas of a patient's body

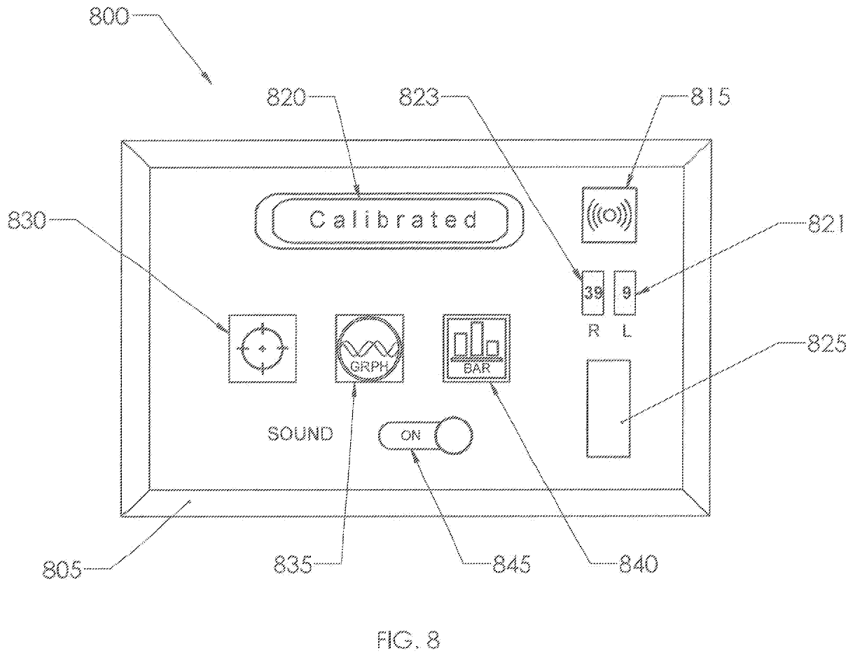

Patient support and positioning base 250 supports the patient 205 during treatment and includes a moveable (e.g., slidable) pallet 255. At least one end of pallet 255 can accept attachment of adjustable support arm 235, which can be adjusted to position attached patient monitor 240 within the patient 205 field of vision. The WSM 230 can provide raw or processed pitch (i.e., angular pitch) data from the IMU to the patient monitor (PM) 240 for processing and depicting as a graph or moving bar indicating relative movement of the patient 205 falling either within or exceeding acceptable parameters. Alternatively, the PM 240 can receive processed data from a sensor control and monitoring unit (SCMU), which can perform all the data processing of the movement data. A sensor control and monitoring unit (SCMU) (not shown, but see FIG. 8) can receive data from the WSM 230 and process the data for display. The measured variables and established parameters can include consideration of both patient position and time duration.

In some embodiments, instead of an oncology treatment unit, a X-ray or other imaging unit such as an MRI may be in use.

FIG. 3 shows an embodiment of a radiation therapist control work station 300. The work station 300 comprises multiple GUI displays 310a, 310b, 310c, and 310d monitored by a radiation therapist 305. A standard computer keyboard 315 and specialized control input keyboard 320 along with a control interface 325 can be used to control the patient support and positioning base 250 as well as the oncology treatment unit 210. A laptop computer 330 can also be utilized for treatment monitoring and control. In an exemplary embodiment, a control and monitoring interface can be enabled on the laptop to function as the sensor control and monitoring unit (SCMU) 330 or on the main computer by activating a patient movement monitor application (PMM) stored in memory and executable upon the computers as shown in FIG. 1 and FIG. 3. In another exemplary embodiment, a dedicated and independent sensor control and monitoring unit (SCMU) (not shown) can be used, In such a dedicated embodiment, the SCMU can consists of a 12×12×4 cm case with a 7×5 cm touchscreen. The electronics inside the case can consist of a circuit board, a RFM69 radio, a microcontroller, and a 2500 mAh rechargeable LiPo battery. Regardless of the mode of implementation, the SCMU 330 can be used to interface with and control both the WSM 230 and the PM 240 via wired or wireless connections. In either implementation, the radiation therapist 305 can interface with WSM 230 to control the WSM 230, receive movement data on patient 205, and display data on a GUI (on the PM 240, the SCMU 330, or another work station display). Typically, it is contemplated that the radiation therapist 305 will utilize the SCMU 330 to view the patient feedback data in isolation of other GUI data streams used on legacy systems, while PM 240 will display the patient feedback data to the patient 205. Furthermore, typical construction of a treatment facility includes a radiotherapy vault with thick walls shielding machine operators and therapists in a control room from the oncology treatment unit 210. The thick walls can block wireless signals necessitating locating an antenna or WiFi receiver wirelessly linked to PM 240 and/or WSM 230 in the radiotherapy vault linked by a wired connection to work station 300.

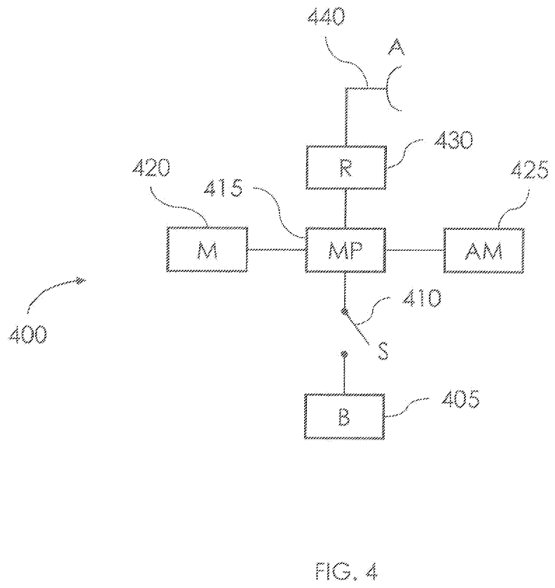

FIG. 4 depicts an electronic circuit for an embodiment of the WSM 230. As depicted, WSM 400 can include a battery (B) 450 that provides power to the circuit. A microprocessor/microcontroller (MP) 415 can operate and control the WSM 230 by executing software. A switch (S) 410 interposed between the MP 415 and B 405 can selectively be used to power on or power off WSM 230. Memory (M) 420 can store executable computer code utilized by the MP 415 to enable WSM 230 to measure movement data of a patient 205. An accelerometer (AM) 425 (more generically an IMU) can be used to measure the movement, with electronic signal data reflecting those movements to the MP 415. Executing the computer code stored in memory M 420, the MP 415 can process the signal data for transmitting to the SCMU using radio (R) 430 to transmit the data using antenna 440.

The MP 415 can execute computer code in association with the SCMU 330 to calibrate the AM 425, process signal data from the AM 425, and generate movement data representing movement meeting positional thresholds and allowing for simple and easy to interpret visual graphics representing movement within treatment parameter and movement outside treatment parameters. In an embodiment, the PM 240 can likewise or alternatively receive data from WSM 230/MP 415 and process the data to generate the visual graphics indicating movement within parameters and movement outside parameters. Further, PM 240 can interface with the SCMU 330, so that the radiation therapist can control and monitor the function of PM 240.

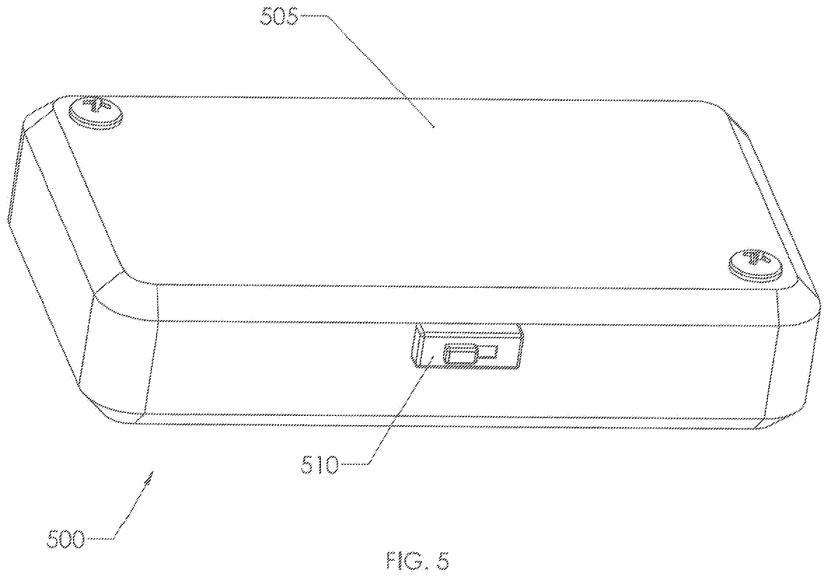

FIG. 5 shows a further embodiment of the exterior of WSM 230. WSM sensor 500 includes a plastic housing 505 containing the electronics of FIG. 4. Switch 510 correlates with S 410. WSM sensor 500 can further feature a bonding surface (not shown) that can be used to attach WSM sensor 500 to a patient 205. Medical adhesive can be used to directly attach the WSM sensor 500 to patient 205, or the bonding surface can include an intermediary structure, such as medical tape or an adhesive strip of a hook and loop fixture (Velcro™).

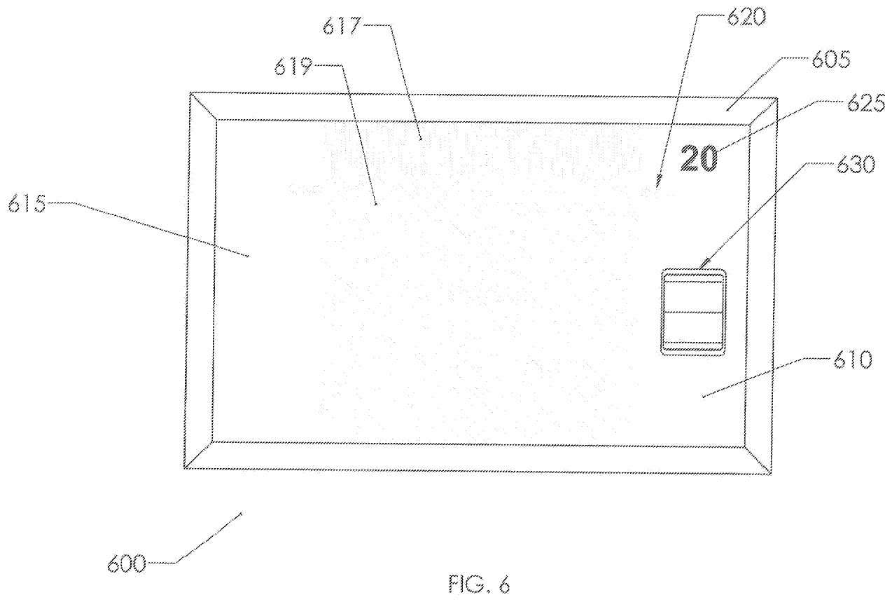

FIG. 6 depicts a displayed graphic in an embodiment for visual display on either PM 240, the SCMU 330, or at work station 300 on a GUI display (e.g., GUI displays 310a, 310b, 310c, and 310d monitored by a radiation therapist 305). As stated earlier, PM 240 or SCMU 330 can comprise a tablet computer. For simplification, the description herein will be directed primarily to a PM 240, but applies equally to the SCMU 330 and/or GUI displays 310a, 310b, 310c, and 310d.

As depicted, a patient monitor (PM) 605 uses a display 610 to provide a two-part graphical data output 615 of processed movement data. The blue upper field 617 on the upper half of the graphical data output is essentially a “filler” default field, while the green lower field 619 on the lower half indicates relative movement of the patient 205. The green standard stubs 620 on either side of the graphical data output 615 represents a movement threshold that meets acceptable movement parameters for a medical procedure. The green color of lower field 619 provides a readily observed indicator that a patient 205 meets acceptable positional parameters, and the parameter indicator 630 provides a secondary indicator that movement is not excessive and meets allowable parameters for the procedure. Breath hold duration 625 is the duration of time that the patient 205 has been able to hold their breath and/or maintain their body position within parameters.

In an embodiment, the PM 240, 605 can autonomously process and display movement data from the WSM 230, or alternatively, the PM 240, 605 can receive and display movement data processed and transmitted by the SCMU 330. Furthermore, the WSM 230 can be sensitive to patient movement from multiple parts of a patient body.

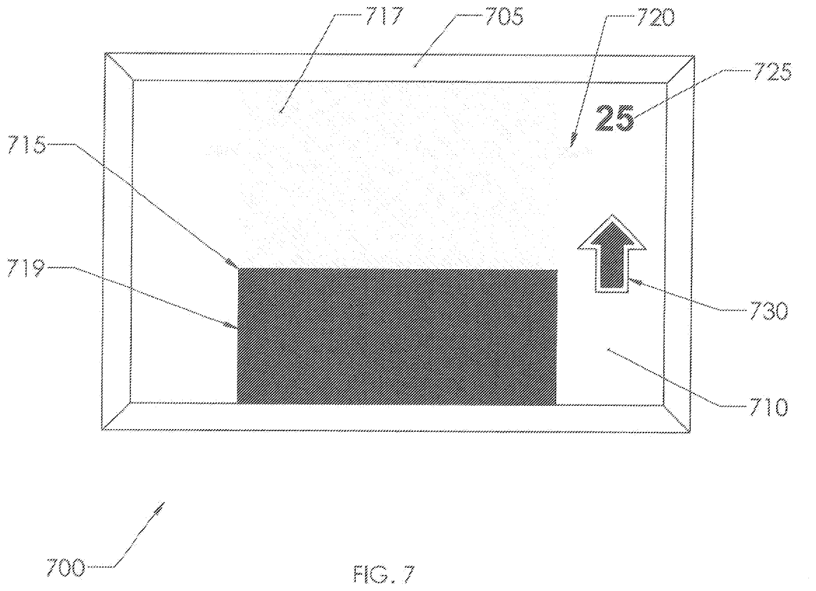

FIG. 7 shows an exemplary embodiment of the displayed graphic in an embodiment for visual display on PM 240, the SCMU 330, or at work station 300 on a GUI display (e.g., GUI displays 310a, 310b, 310c, and 310d) monitored by a radiation therapist 305 with excessive movement by a patient 205. As depicted, a monitor device 705 uses a display 710 to provide a two-part graphical data output 715 of processed movement data. The blue upper field 717 on the upper half of the graphical data output is essentially a “filler” default field, while the black lower field 719 on the lower half indicates relative movement of the patient 205. The green standard stubs 720 on either side of the graphical data output 715 represents a movement threshold that meets acceptable movement parameters for a medical procedure. The black color of lower field 719 provides a readily observed indicator that a patient 205 does not meet acceptable positional parameters, and the breathing prompt arrow 730 cues patient 205 to either breathe in (up arrow) or breathe out (down arrow), and can provide a secondary indicator that movement meets or exceeds allowable parameters for the procedure. Breath hold duration 725 is the duration of time that the patient 205 has been able to hold their breath and/or maintain their body position within parameters. Of course, different display color combinations can further aid in graphically indicating movement within, movement exceeding, or movement minimally within parameters (e.g., red, amber, black, green, orange, white, etc).

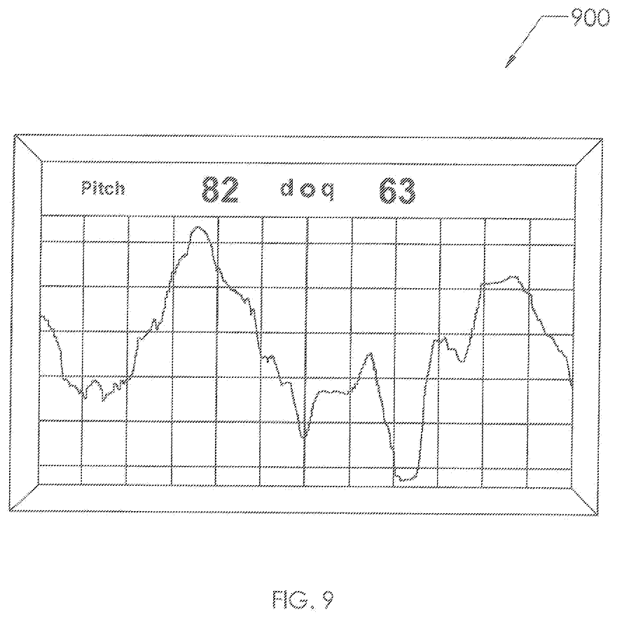

FIG. 8 depicts an embodiment of a dedicated SCMU 800 used to interface with and control WSM 230. SCMU 800 comprises an outer body 805 and uses a touch sensitive display screen 810. As depicted, SCMU 800 communicates over a wireless signal with PM 240 and WSM 230, and a signal status indicator 815 indicates a good connection with the PM 240 and WSM 230. Calibration icon 820 can be used to initiate calibration of WSM 230, and the patient 205 may be directed to make various movements during this calibration process. Battery charge indicators 821 and 823 indicate the battery charge in PM 240 (i.e., remote battery) and SCMU 800 (i.e., local battery) respectively. Movement parameter indicator 825 can provide a graphic indication of movement. Target touch icon 830 can initiate the calibration process for the system. Graph touch icon 835 can select a display mode of a line graph of processed movement data as depicted in FIG. 9. Bar touch icon 840 can select a display mode of a bar graph of processed movement data. Sound touch icon 845 can activate or deactivate a generate sound alarm function to alert when movement exceeds parameters and/or alert when movement is within allowed parameters.

In an embodiment, the SCMU 800 can be used to control what data is displayed on PM 240 and/or share data and display graphics on PM 240. Of course, alternatively, PM 240 can be independently configured to process movement data received from the WSM 230 and display the data, and/or the SCMU 800 can be used to configure or change the configuration of the PM 240 remotely, to include transmitting and sharing the graphic displays on the SCMU 800.

FIG. 9 depicts a graph of processed movement data. As shown, the movement data can be displayed as a graph of angular pitch data provided by the WSM 230 over time. In an embodiment, a share data configuration can be selected to display the same view on the PM 240 and the SCMU 330 or any other display on the radiological oncology treatment system such as GUI displays 310a, 310b, 310c, and 310d monitored by a radiation therapist 305.

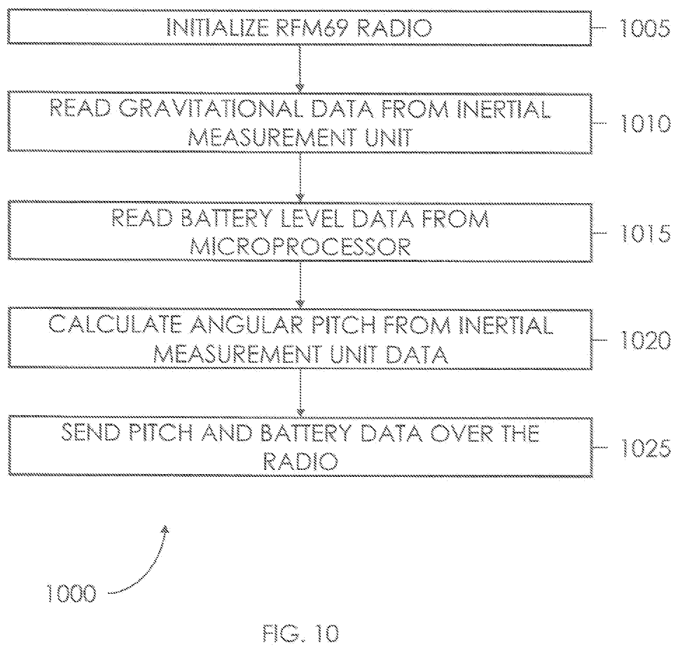

In an embodiment, FIG. 10 shows a breathing monitor flowchart depicting a process flow for the WSM 230. In step 1005, the microprocessor in WSM 230 executes PPM software stored in memory to initialize the radio transceiver. In step 1010, the microprocessor reads gravitational data generated by and received from the inertial measurement unit. In step 1015, the WSM 230 reads battery level data from the microprocessor. The microprocessor calculates angular pitch data from inertial measurement unit data in step 1020, and the microprocessor then sends pitch and battery data over the radio in step 1025.

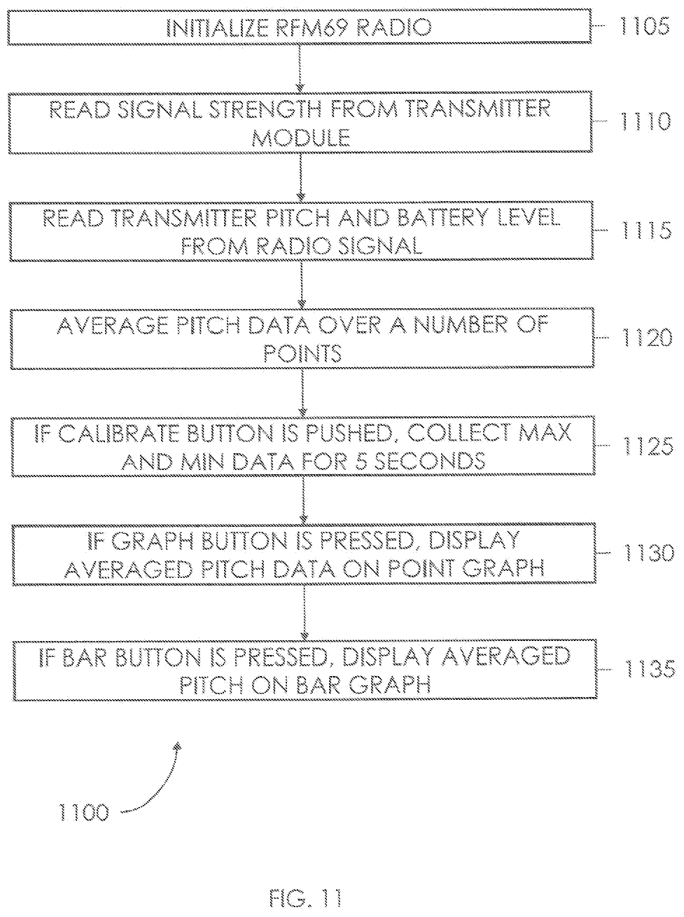

In an embodiment, FIG. 11 depicts a receiver module flowchart depicting a process flow for SCMU 800. In step 1105, the microprocessor in SCMU 800 executes PPM software stored in memory to initialize the radio transmitter. In step 1110, the SCMU 800 reads signal strength from the transmitter WSM 230. In step 1120, the SCMU 800 averages pitch data over a number of data points. If calibration icon 820 is activated, the SCMU 800 collects maximum and minimum data from WSM 230 for five seconds in step 1125. In step 1130, if the graph icon 835 is activated, display 810 displays averaged pitch data on a point/line graph. In step 1135, if bar icon 840 is activated, display 810 displays averaged pitch data on a bar graph.

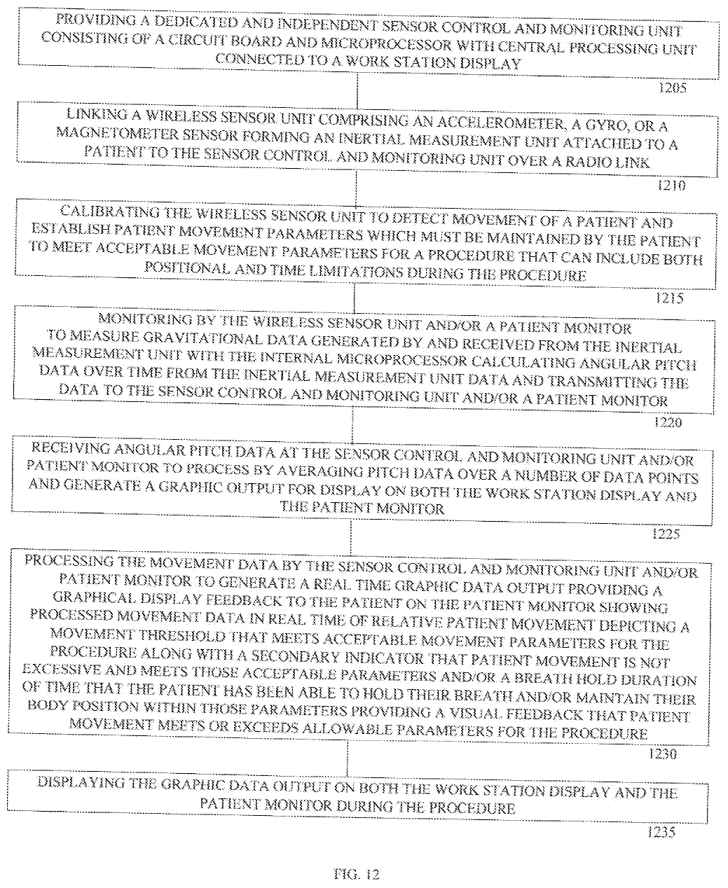

FIG. 12 depicts an exemplary process flowchart showing the general overall process for using the sensor control and monitoring unit interfaced with the wireless sensor module to generate and output a patient feedback graphic onto a patient monitor. In step 1205, the process starts by providing a dedicated and independent sensor control and monitoring unit consisting of a circuit board and microprocessor with central processing unit connected to a treating display visible to a medical personnel. Step 1210 consist of linking a wireless sensor unit comprising an accelerometer, a gyro, or a magnetometer sensor forming an inertial measurement unit and attached to a patient to the sensor control and monitoring unit over a radio link. In step 1215, the wireless sensor unit is calibrated to detect movement of a patient and establish patient movement parameters which must be maintained by the patient to meet acceptable movement parameters for a procedure that can include both positional and time limitations during a procedure. This can include directing the patient to make various movements during the calibration process to establish these movement parameters. Step 1220 requires monitoring by the wireless sensor unit to measure gravitational data generated by and received from the inertial measurement unit with the internal microprocessor calculating angular pitch data over time from the inertial measurement unit data and transmitting the data to the sensor control and the monitoring unit and/or a patient monitor.

In step 1225, the sensor control and monitoring unit or patient monitor receives angular pitch data to process by averaging pitch data over a number of data points and generate a graphic output for display on both the work station display and the patient monitor (e.g. GUIs). In step 1230, the sensor control and monitoring unit or patient monitor processes the movement data to generate a real time graphic data output providing a graphical display feedback to the patient showing processed movement data in real time of relative patient movement depicting a movement threshold that meets acceptable movement parameters for the procedure along with a secondary indicator that patient movement is not excessive and meets those acceptable parameters and/or a breath hold duration of time that the patient has been able to hold their breath and/or maintain their body position within those parameters to provide a visual feedback that patient movement meets or exceeds allowable parameters for the procedure. In step 1235, both the work station display and the patient monitor display the graphic data output during the medical procedure (i.e., in real time).

The algorithm and necessary algorithms and computer code to convert the inertial measurement unit data with the internal microprocessor to calculate angular pitch data is well known within the art. Exemplary technical examples of this usage and data conversion can be found on the Internet as follows:
https://arxiv.org/pdf/1704.06053.pdf
https://ietresearch.onlinelibrary.wiley.com/doi/10.1049/iet-smt.2016.0459
https://www.vectornay.com/resources/inertial-navigation-articles/what-is-an-inertial-measurement-unit-imu
https://itp.nyu.edu/physcomp/lessons/accelerometers-gyros-and-imus-the-basics/
https://www.mathworks.com/help/fusion/gs/spatial-representation-coordinate-systems-and-conventions.html
https://www.mathworks.com/help/fusion/gs/determine-orientation-through-sensor-fusion.html
http://www.starlino.com/imu_guide.html
OpenSense—Kinematics with IMU Data—OpenSin Documentation—Global Site (stanford.edu)

The inventor has written the following exemplary code to run on the SCMU to implement the invention and display angular pitch and breathing data for display:

The inventor has written the following exemplary code to run on the WSM to implement the invention and converts IMU data into angular pitch data for display:

```
The following Arduino code is what runs the Wireless sensor module. It is compiled
in the Arduino IDE, then uploaded through its serial port and resides in it's memory.
Comments appear either between /* and */ or after two front slashes “//”
/*
  * code to read IMU data and send over RFM69 radio
* written by A.Markovic for Adafruit Faether M0 Express connected to a 9 degree
of freedom inertial measurement unit
datasheet: https://cdn-shop.adafruit.com/datasheets/LSM303DLHC.PDF
  */
//the lines below call libraries that are needed to run the algorithm
#include <stdlib.h> //standard arduino library
#include “dtostrf.h” //library to convert decimal to string
#include <Wire.h> //allows communication through the i2c pins on the
Microcontroller
#include <RFM69.h> //allows communication through a wireless protocol
#include <SPI.h> //allows communication through SPI pins on CPU
#include <Adafruit_LSM303_Accel.h> //provides commands to read
accelerometer data
#include <Adafruit_Sensor.h> // not used in this demo but required!
/* Assign a unique ID to this sensor at the same time */
Adafruit_LSM303_Accel_Unified accel =
Adafruit_LSM303_Accel_Unified(54321);
// The following commented lines are used to display certain data for
troubleshooting purposes
/*
void displaySensorDetails(void) {
  sensor_t sensor;
  accel.getSensor(&sensor);
  Serial.println(“------------------------------------”);
  Serial.print(“Sensor: ”);
  Serial.println(sensor.name);
  Serial.print(“Driver Ver: ”);
  Serial.println(sensor.version);
  Serial.print(“Unique ID: ”);
  Serial.println(sensor.sensor_id);
  Serial.print(“Max Value: ”);
  Serial.print(sensor.max_value);
  Serial.println(“ m/s\hat{ }2”);
  Serial.print(“Min Value: ”);
  Serial.print(sensor.min_value);
  Serial.println(“ m/s\hat{ }2”);
  Serial.print(“Resolution: ”);
  Serial.print(sensor.resolution);
  Serial.println(“ m/s\hat{ }2”);
  Serial.println(------------------------------------”);
  Serial.println(“”);
  delay(500);
}
*/
float x = 0.0; //assign floating point variables x,y
float y = 0.0;
// i2c
#define NETWORKID 100 // The same on all nodes that talk to each other
#define NODEID 2 // The unique identifier of this node
#define RECEIVER 1 // The recipient of packets
//Match frequency to the hardware version of the radio on your microprosser. Sets
wireless communication to 915 MHz
//#define FREQUENCY RF69_433MHZ
//#define FREQUENCY RF69_868MHZ
#define FREQUENCY RF69_915MHZ
//#define ENCRYPTKEY “sampleEncryptKey” //exactly the same 16
characters/bytes on all nodes!
#define IS_RFM69HCW true // set to ‘true’ if you are using an RFM69HCW
module
//****************************************************************
*****************************
#define SERIAL_BAUD 115200
#define VBATPIN A7
//for feather (microprocessor) communication parameters:
#define RFM69_CS 8
#define RFM69_IRQ 3
#define RFM69_IRQN 0 // Pin 3 is IRQ 0!
#define RFM69_RST 4
RFM69 radio = RFM69(RFM69_CS, RFM69_IRQ, IS_RFM69HCW,
RFM69_IRQN);
//Define variables used in the algorithm
char pitchStr[10];
char AccelzStr[10];
char vbatstr[10];
char buffer[60];
int count = 0;
float ax; //acceleration in x-direction
float ay;
float az;
float pitch; //define pitch as a flaoting point number
float cal = −4; //degrees used to calibrate the angle from the pitch calculation
float accelX, accelY, accelZ, accelR;
void setup( )
{
  Serial.begin(SERIAL_BAUD);
//readings[1]=100.1; //initialize array
  // while (!Serial) {
  // delay(1); // will pause Zero, Leonardo, etc until serial console opens
// }
    Serial.println(“LSM9303 data read demo”);
    // Try to initialize and warn if can't detect the chip
  if (!accel.begin( ))
  {
  Serial.println(“Oops ... unable to initialize the LSM9303. Check your wiring!”);
  while (1);
  }
  // displaySensorDetails( );
  //set g range to =/− 2G
  accel.setRange(LSM303_RANGE_2G);
    accel.setMode(LSM303_MODE_HIGH_RESOLUTION);
    //reset radio// Hard Reset the RFM module
  pinMode(RFM69_RST, OUTPUT);
  digitalWrite(RFM69_RST, HIGH);
  delay(100);
  digitalWrite(RFM69_RST, LOW);
  //delay(100);
// Initialize radio
  radio.initialize(FREQUENCY,NODEID,NETWORKID);
  if (IS_RFM69HCW) {
  radio.setHighPower( ); // Only for RFM69HCW & HW!
  }
  radio.setPowerLevel(31); // power output ranges from 0 (5dBm) to 31 (20dBm)
  // radio.encrypt(ENCRYPTKEY);
  //send parameters over serial port for troubleshooting and monitoring
  Serial.print(“\nTransmiţng at ”);
Serial.print(FREQUENCY==RF69_433MHZ ? 433 :
FREQUENCY==RF69_868MHZ ? 868 : 915);
  Serial.println(“ MHz”);
  Serial.println(“ X Y Z R”);
  }
byte sendLen;
void loop( )
{
float measuredvbat = analogRead(VBATPIN); //read and renormalize battery level
data
measuredvbat *= 2; // we divided by 2, so multiply back
measuredvbat *= 3.3; // Multiply by 3.3V, our reference voltage
measuredvbat /= 1024; // convert to voltage
//Serial.println(“VBat: ” ); Serial.println(measuredvbat);
//delay(500);
  // char radiopacket[20]; // packet
  // float time = millis( )/1000.0; //time is milli seconds
    sensors_event_t event;
  accel.getEvent(&event);
  //following lines display data and are for troubleshooting
/* Display the results (acceleration is measured in m/s\hat{ }2)
  Serial.print(“X: ”);
  Serial.print(event.acceleration.x);
  Serial.print(“ ”);
  Serial.print(“Y: ”);
  Serial.print(event.acceleration.y);
  Serial.print(“ ”);
  Serial.print(“Z: ”);
  Serial.print(event.acceleration.z);
  Serial.print(“ ”);
  Serial.println(“m/s\hat{ }2”);
  Delay before the next sample */
  // delay(500);
//read acceleration in x, y, z -directions from inertial measurement unit
(accelerometer)
ax=event.acceleration.x;
ay=event.acceleration.y;
az=event.acceleration.z;
pitch = atan2(az,ay); //calculate the pitch with the arctangent function using the x
and y accelerations
// Convert everything from radians to degrees:
pitch = abs(pitch*(180.0 / PI) + cal);
//send the acceleration data in all three directions to the serial port
Serial.print(“ax = ”);Serial.print(ax);
Serial.print(“ ay = ”);Serial.print(ay);
Serial.print(“ az = ”);Serial.println(az);
//Serial.print(“ pitch = ”);Serial.println(pitch,4);
  //convert to string data for transmission
dtostrf(measuredvbat,3,1,vbatstr);
dtostrf(pitch,4,1,pitchStr);//convert the float to string -
dtostrf(FLOAT,WIDTH,PRECISION,BUFFER);
dtostrf(az,4,1,AccelzStr);
//delay(100);
//put the pitch, z-acceleration and battery voltage into a single data element called
“buffer”
sprinn(buffer,“P:%s Accelz: %s Batt: %s”, pitchStr,AccelzStr,vbatstr);
sendLen=strlen(buffer); //calculate the length of the data element
//delay(100);
//for(int i=0; i<4; i++){ //this loop fills the packet with the time parameter
// radiopacket[i]=timeS[i];
//}
//send the data element over the radio in the wireless sensor module (WSM)
radio.send(RECEIVER, buffer, sendLen); //target node Id, message as string or
byte array, message length
//Serial.println(“OK”);
//Serial.println(buffer);
blink(13,5);
  Serial.flush( ); //make sure all serial data is clocked out before sleeping the MCU
  // delay(200);
  radio.receiveDone( ); //put radio in RX mode
}
//the following subroutine blinks the light on the WSM when data is being sent
void blink(byte PIN, byte DELAY_MS)
{
  pinMode(PIN, OUTPUT);
  digitalWrite(PIN,HIGH);
  delay(DELAY_MS/2);
  digitalWrite(PIN,LOW);
  delay(DELAY_MS/2);
}
```

The implemented system can monitor the movement of a patient and display the data in an easily understood format to inform a patient that their movements remain within acceptable parameters or exceed those acceptable parameters for a medical procedure and can greatly aid in keeping patients in position. A double display output for both the patient and the medical equipment operator to monitor movement can offer considerable flexibility of use and improve patient compliance. The disclosed small and simple movement sensor can further improve ease of use and patient comfort. Thus, the implemented invention as used in the medical imaging and radiology oncology treatment arts can facilitate increased compliance by patients to keep body movement within acceptable parameters. A simple, easy to use sensor and data monitor and display system enables individual patients to comply with instructions to minimize movement and remain positioned correctly for medical procedures, which can include breath hold durations and thresholds, and it provides dual real time movement data to both the patient and the equipment operator.

Insofar as the description above and the accompanying drawing disclose any additional subject matter that is not within the scope of the single claim below, the inventions are not dedicated to the public and the right to the one or more applications to claim such additional inventions is reserved.

Of course the present invention is not intended to be restricted to any particular form or arrangement, or any specific embodiment, or any specific use, disclosed herein, since the same may be modified in various particulars or relations without departing from the spirit or scope of the claimed invention hereinabove shown and described of which the apparatus or method shown is intended only for illustration and disclosure of an operative embodiment and not to show all of the various forms or modifications in which this invention might be embodied or operated.

The foregoing has described methods and systems for a patient movement monitoring and feedback system that are given for illustration and not for limitation and uses. Thus, the inventions are limited only by the appended claims. Although the inventions have been described in accordance with the embodiments shown, one of ordinary skill in the art will readily recognize that there could be variations to the embodiments and those variations would be within the spirit and scope of the present inventions. Accordingly, many modifications may be made by one of ordinary skill in the art without departing from the spirit and scope of the appended claims.

Particular terminology used when describing certain features or aspects of the embodiments should not be taken to imply that the terminology is being redefined herein to be restricted to any specific characteristics, features, or aspects with which that terminology is associated. In general, the terms used in the following claims should not be construed to be limited to the specific embodiments disclosed in the specification, unless the above Detailed Description section explicitly defines such terms. Accordingly, the actual scope of the claims encompasses not only the disclosed embodiments, but also all equivalent ways of practicing or implementing the claimed subject matter.

The above detailed description of the embodiments is not intended to be exhaustive or to limit the disclosure to the precise embodiment or form disclosed herein or to the particular fields of usage mentioned above. While specific embodiments and examples are described above for illustrative purposes, various equivalent modifications are possible within the scope of the disclosure, as those skilled in the relevant art will recognize. Also, the teachings of the embodiments provided herein can be applied to other systems, not necessarily the system described above. The elements and acts of the various embodiments described above can be combined to provide further embodiments.

Any patents, applications and other references that may be listed in accompanying or subsequent filing papers, are incorporated herein by reference. Aspects of embodiments can be modified, if necessary, to employ the systems, functions, and concepts of the various references to provide yet further embodiments.

In light of the above “Detailed Description,” the Inventors may make changes to the disclosure. While the detailed description outlines possible embodiments and discloses the best mode contemplated, no matter how detailed the above appears in text, embodiments may be practiced in a myriad of ways. Thus, implementation details may vary considerably while still being encompassed by the spirit of the embodiments as disclosed by the inventors. As discussed herein, specific terminology used when describing certain features or aspects should not be taken to imply that the terminology is being redefined herein to be restricted to any specific characteristics, features, or aspects of the embodiments with which that terminology is associated.

While certain aspects are presented below in certain claim forms, the inventors contemplate the various aspects in any number of claim forms. Accordingly, the inventors reserve the right to add additional claims after filing the application to pursue such additional claim forms for other aspects.

The above specification, examples and data provide a description of the structure and use of exemplary implementations of the described systems, articles of manufacture and methods. It is important to note that many implementations can be made without departing from the spirit and scope of the disclosure.

### MODIFICATIONS AND VARIATIONS

As will be recognized by those skilled in the art, the innovative concepts described in the present application can be modified and varied over a tremendous range of applications, and accordingly the scope of patented subject matter is not limited by any of the specific exemplary teachings given. It is intended to embrace all such alternatives, modifications and variations that fall within the spirit and broad scope of the appended claims.

The various computer component of the system can be implemented using computer code to configure various wireless communication devices incorporating a memory and an associated microprocessor. Existing wireless devices can be configured to serve as the hardware components of the system such as the PM 240 and the WSM 230.

The WSM can be implemented using a wired movement sensor connected to a communication device. Furthermore, the system can be scaled upwards if required to use multiple WSMs attached to various body parts.

None of the description in the present application should be read as implying that any particular element, step, or function is an essential element which must be included in the claim scope: THE SCOPE OF PATENTED SUBJECT MATTER IS DEFINED ONLY BY THE ALLOWED CLAIMS. Moreover, none of these claims are intended to invoke paragraph six of 35 USC section 112 unless the exact words “means for” are followed by a participle.

The claims as filed are intended to be as comprehensive as possible, and NO subject matter is intentionally relinquished, dedicated, or abandoned.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. An apparatus for monitoring patient movement during a medical procedure, comprising:
a single calibrated wireless sensor module comprising an internal microprocessor with a CPU coupled to an associated first memory and first radio responsive to movement using an inertial measurement unit measuring angular pitch changes consisting of measuring gravitational data generated by the inertial measurement unit with the internal microprocessor calculating angular pitch data over time;
a patient movement monitor comprising a display with processed patient movement data displayed including visual data graphically depicting movement within one or more movement parameters required to execute the medical procedure and visible to a patient during the medical procedure, said patient movement monitor interfaced with the wireless sensor module;
a sensor control and monitoring unit comprising a microprocessor with CPU, an associated second memory, second radio, and associated work station display remotely interfaced with the wireless sensor module to control the wireless sensor module and to receive movement data as calculated angular pitch data over time, process the movement data by averaging pitch data over a number of data points and generate a graphic output on an associated display, and display the processed movement data as a graphic indicating movement within at least one movement parameter, said sensor control and monitoring unit connected to the patient movement monitor and the work station display;
wherein the sensor control and monitoring unit processes the movement data to generate a real time graphic data output providing a graphical display feedback to the patient movement monitor to the patient showing processed movement data in real time of relative patient movement depicting a movement threshold that meets acceptable movement parameters for the medical procedure along with a secondary indicator that patient movement is not excessive and also meets the acceptable movement parameters and/or 1) a breath hold duration of time that the patient has been able to hold their breath and/or 2) time the patient has maintained their body position within the acceptable movement parameters, whereby said patient movement monitor provides a visual feedback to the patient that patient movement meets or exceeds the acceptable movement parameters for the medical procedure, both said patient movement monitor and work station display displaying the real time graphic data output.

21. The apparatus for monitoring patient movement of claim 20, wherein the wireless sensor module further comprises a microprocessor and executable computer code stored in the associated memory with said inertial measurement unit connected to the microprocessor and sensitive to angular pitch changes of a patient when attached to said patient.

22. The apparatus for monitoring patient movement of claim 20, wherein the patient movement monitor further comprises a microprocessor and executable computer code stored in an associated memory.

23. The apparatus for monitoring patient movement of claim 20, wherein the sensor control and monitoring unit further comprises a microprocessor and executable computer code stored in the associated memory.

24. The apparatus for monitoring patient movement of claim 20, wherein the patient movement monitor receives pitch data from the wireless sensor module and autonomously processes this movement data by averaging pitch data over the number of data points to display as a graphic indicating movement within at least one movement parameter to display to the patient.

25. The apparatus for monitoring patient movement of claim 20, wherein the wireless sensor module collects data sensitive to movement of multiple areas of a patient body.

26. The apparatus for monitoring patient movement of claim 20, wherein the wireless sensor module attaches to a patient body using a medical adhesive.

27. The apparatus for monitoring patient movement of claim 20, wherein the sensor control and monitoring unit comprises a touch sensitive display screen.

28. The apparatus for monitoring patient movement of claim 20, wherein the sensor control and monitoring unit comprises a control input for selecting a graphics display mode.

29. The apparatus for monitoring patient movement of claim 20, wherein the sensor control and monitoring unit comprises a control input for calibrating the wireless sensor module.

30. The apparatus for monitoring patient movement of claim 20, wherein the sensor control and monitoring unit comprises a control input for sound.

31. The apparatus for monitoring patient movement of claim 20, wherein the sensor control and monitoring unit comprises a status indicator for at least the wireless sensor module.

32. The apparatus for monitoring patient movement of claim 20, further comprising a non-transitory tangible storage media storing computer code for configuring at least one of said wireless sensor module, patient movement monitor, and sensor control and monitoring unit to monitor patient movement; said patient movement monitor further comprising a CPU.

33. A system for monitoring patient movement during a medical procedure, comprising:
a single calibrated wireless sensor module comprising an internal microprocessor with a CPU coupled to an associated first memory and first radio with an inertial measurement unit measuring angular pitch changes to calculate angular pitch data over time;
a patient movement monitor comprising a display with processed patient movement data displayed including visual data graphically depicting compliance and/or non-compliance with one or more acceptable movement parameters required to execute the medical procedure and visible to a patient during the medical procedure;
a sensor control and monitoring unit comprising a microprocessor with CPU, an associated second memory, second radio, and associated work station display remotely interfaced with the wireless sensor module to control the wireless sensor module and to receive movement data as calculated angular pitch data over time, process the movement data by averaging pitch data over a number of data points and generate a graphic output on an associated display, and display the processed movement data as a graphic indicating movement within the one or more acceptable movement parameters, said sensor control and monitoring unit connected to the patient movement monitor and the work station display;
wherein the sensor control and monitoring unit processes the movement data to generate a real time graphic data output providing a graphical display feedback to the patient movement monitor showing processed movement data in real time of relative patient movement depicting at least one acceptable movement threshold that meets the one or more acceptable movement parameters for the medical procedure and an indication that patient movement meets the one or more acceptable movement parameters, whereby said patient movement monitor provides a visual feedback comprised of the real time graphic data output to the patient indicating the patient movement meets or exceeds the one or more acceptable movement parameters for the medical procedure, both said patient movement monitor and work station display displaying the real time graphic data output.

34. The system of claim 33 further comprising a graphic depiction on the real time graphic indicating at least one of:
a breath hold duration of time that the patient has been able to hold their breath; or
time duration the patient has maintained their body position within at least one acceptable movement parameter.

35. The system of claim 33 wherein the medical procedures comprises at least one of an imaging procedure or an oncology/radiation treatment.

36. The system of claim 33 wherein the work station display comprises a work station that further comprises the sensor control and monitoring unit.

37. A system for monitoring patient movement during a medical procedure requiring minimal movements, comprising:
a wireless sensor module comprising an inertial measurement unit sensitive to movement and generating movement data consisting of derived angular pitch data calculated by an internal microprocessor, said wireless sensor module attached to a patient;
a patient movement monitor interfaced with the wireless sensor module displaying processed angular pitch data including a real time graphic data output depicting movement within one or more movement parameters required to execute the medical procedure and visible to the patient during the medical procedure;
a sensor control and monitoring unit in communication with the wireless sensor module and/or the patient movement monitor to control the wireless sensor module and/or patient movement monitor to receive the movement data, process the movement data, and display the movement data as the real time graphic indicating movement within one or more movement parameter with a dual output of the real time graphic data output to an associated display visible at a work station and on the patient movement monitor.

38. The system for monitoring patient movement of claim 37, wherein the patient movement monitor autonomously receives and processes movement data to display as the real time graphic data output.

39. The system for monitoring patient movement of claim 37, wherein the processed movement data in the graphic data output comprises one or more movement thresholds for the medical procedure.
